SEQUENCE LISTING (49 sequences)

SEQ ID NO: 1 - DNA, 1527 bp, synthetic construct
  source 1..1527: mol_type = other DNA
atggatgcaatagtattccacccctatgttttccaagcccttggatctctcctcatcttatggctgatttcaaaatggttcaacaaatccaatggaaccaagaacttgcccccgtcaccgccgatgctcccgataatcggaaacctccaccagctgagctcattgcctcaccacgacctccggcgcttggcgcgcaaacatggtcctctcatgctgctgcactttggcaccgtgcctacgctcatcgtgtcatccgtggagggcgccaaggaaataatgaaaactcatgatcttgcatttgcagaccggcccgagtcttgggttactaagaggctgctctacgatcacaaggatgtgtcggttgcgccgtatggcgaatattggagacagttgaagagtatatgtgtgctccagcttctcagcaataaaagagtccaatctttccattcgatccgggaagaagaaatgtcccttttgatggaaaaaattaggggctcgtcacgcgcgttgaatttgagtgagatgttcactaaagtcactaatgatatggtttgtagatcggcgtttggtgtgaaatacggcgagggagaaaaggggaagaaattcatggcgcttttgacggagtttttggagttgttagggaccatttacatcggaaattttgtgccgtggctttcgtggattactcgtcttaccgggttcgatgcaagagtggataaggttgcaaaagagctcgacatattgatggaggatgtgatcgaagaaaggattagaagaaatcttgaagataaggctgagaatgaacacaagtatggacaaaattttgtagatattttggtagatatttacaagaacaattctgcaggcatctccatggacagagatagtgtgaaagcaatacttttggatgtttttgcagctgggacggacaccacatcgactgttctagaatgggcgatgagtgaactccttcgacaccccgaggtgatgaagaaactgcaaaatgaagtgaggggagtaatgaaagacaagggcaagataagcgatgaagacttagaaaaaatgcaatatctaaaagccgtgatcaaagaaactcttcgcctgcacactccgatcccagctctagttcctcggatggcaagaaaagatgtcaaggttatgggatacgatgtatcagccgggacaatggtgatgattaatgcttgggccataggcagagaccccggcatttgggatgagcctgaaaagttcaaccctaaccggtttctaaattcatcgatagatttcaagggccaggatttcgagctgatcccttttggggccgggcggcgaggctgcccggggatttcatttgctatggctaccaatgagtttgtgttagcaaatattgtgcataacttcaagtggaaattggctgatgatgagaaagaattagacatgagtgaacgccccggtgtcgccattcgtagggctgttcctcttcttgccgtggcatcaaagttctatactgagggtgtgttcgtttga

SEQ ID NO: 2 - DNA, 1515 bp, synthetic construct
  source 1..1515: mol_type = other DNA
atggatcgtgatttcttatcttctattatccttttttcttctctcatatttcttgcattcttatggaatctaatcactcgctccaaacgtccccggagttccggcgaacttccgccggggccgagaaagctgccggtcatcgggaacttgcacaaccttgtcagctcccgaccaccgcatcatattttagccgacttggcgtcgaaatatggggcgatgatgcacttgcggctaggtgaaatcagcacggttgttatatcgtcgccggaggcagcaaaagaggtcatgaagacatatgacatcaacttcgctaataggccatctttctatgcatcgaagattataacttatgacaactccgacataggattctccaatggagaataccggaaaaatttgcgaaaagtaagcaaaatggagctgctgagtgcgaagcgcgtcaaatcttttcgtcccttaagggaagaagtgttttcggatatgtgcagttggatagcttcaaaagaaggctcgtcgataaacttgactgagaaagtttccttggcgacttttgatgtggctttgcgggcatcgcttggcggaaagattgatgaacacacagcaatggtagatgtaactaaggagtctttcgatttactcgcaggattttatgccggcgatttgtttccctccatcagtttgttgcaatcaatcgacggatttagaggtcgtgtggagaaggtgcacaaactatccgatgccatactccagaagattattgacagtcgaaaagtagccaaatctcaaggcaaaacacacgaaaacttggttgatattctcctcaaattccataaggataccgggcatgatctcggcttaagcgatgacaatataaaagctgtgctacaggatatgttcattgctggaatcgaaacatcatcgacaactacagattgggccatggcagaaatgatgaaacatccaagccttcttaagaaggcacaagatgaggtgagacagatttttctcgacaagggatttgtcgatgagtccgacttcgatgagctaaagtacctaaacttagtgatcaaagaaactttccgaatccacccgccggggcctttaattctcagagaaaacaaagagacctgtgagatcaatggatacacaatacctgcaaaaacaagggttgtggtgaatgcatgggccattggaagagatcccaaaatctggaaagatgccgacagctttataccagagagatttctcgataacaatatttcagccgattacactggaaagaatttcgattacatcccttttggctctggtcgaaggatttgccctggaatgtcattcggcatagctaacgtcgaattcccattggcaatgttactctaccacttcgattgggttttgccacagggaatcaaacctgaagatgtggacatggctgaatcctttggccttacagcagtaagaaaaactcctcttactgtgattcccaaattgaaaaaccctttgcctgtaaagtaa

SEQ ID NO: 3 - DNA, 1431 bp, synthetic construct
  source 1..1431: mol_type = other DNA
accaagaacttgcccccgtcaccgccgatgctcccgataatcggaaacctccaccagctgagctcattgcctcaccacgacctccggcgcttggcgcgcaaacatggtcctctcatgctgctgcactttggcaccgtgcctacgctcatcgtgtcatccgtggagggcgccaaggaaataatgaaaactcatgatcttgcatttgcagaccggcccgagtcttgggttactaagaggctgctctacgatcacaaggatgtgtcggttgcgccgtatggcgaatattggagacagttgaagagtatatgtgtgctccagcttctcagcaataaaagagtccaatctttccattcgatccgggaagaagaaatgtcccttttgatggaaaaaattaggggctcgtcacgcgcgttgaatttgagtgagatgttcactaaagtcactaatgatatggtttgtagatcggcgtttggtgtgaaatacggcgagggagaaaaggggaagaaattcatggcgcttttgacggagtttttggagttgttagggaccatttacatcggaaattttgtgccgtggctttcgtggattactcgtcttaccgggttcgatgcaagagtggataaggttgcaaaagagctcgacatattgatggaggatgtgatcgaagaaaggattagaagaaatcttgaagataaggctgagaatgaacacaagtatggacaaaattttgtagatattttggtagatatttacaagaacaattctgcaggcatctccatggacagagatagtgtgaaagcaatacttttggatgtttttgcagctgggacggacaccacatcgactgttctagaatgggcgatgagtgaactccttcgacaccccgaggtgatgaagaaactgcaaaatgaagtgaggggagtaatgaaagacaagggcaagataagcgatgaagacttagaaaaaatgcaatatctaaaagccgtgatcaaagaaactcttcgcctgcacactccgatcccagctctagttcctcggatggcaagaaaagatgtcaaggttatgggatacgatgtatcagccgggacaatggtgatgattaatgcttgggccataggcagagaccccggcatttgggatgagcctgaaaagttcaaccctaaccggtttctaaattcatcgatagatttcaagggccaggatttcgagctgatcccttttggggccgggcggcgaggctgcccggggatttcatttgctatggctaccaatgagtttgtgttagcaaatattgtgcataacttcaagtggaaattggctgatgatgagaaagaattagacatgagtgaacgccccggtgtcgccattcgtagggctgttcctcttcttgccgtggcatcaaagttctatactgagggtgtgttcgtttga

SEQ ID NO: 4 - DNA, 1430 bp, synthetic construct
  source 1..1430: mol_type = other DNA
aaacgtccccggagttccggcgaacttccgccggggccgagaaagctgccggtcatcgggaacttgcacaaccttgtcagctcccgaccaccgcatcatattttagccgacttggcgtcgaaatatggggcgatgatgcacttgcggctaggtgaaatcagcacggttgttatatcgtcgccggaggcagcaaaagaggtcatgaagacatatgacatcaacttcgctaataggccatctttctatgcatcgaagattataacttatgacaactccgacataggattctccaatggagaataccggaaaaatttgcgaaaagtaagcaaaatggagctgctgagtgcgaagcgcgtcaaatcttttcgtcccttaagggaagaagtgttttcggatatgtgcagttggatagcttcaaaagaaggctcgtcgataaacttgactgagaaagtttccttggcgacttttgatgtggctttgcgggcatcgcttggcggaaagattgatgaacacacagcaatggtagatgtaactaaggagtctttcgatttactcgcaggattttatgccggcgatttgtttccctccatcagtttgttgcaatcaatcgacggatttagaggtcgtgtggagaaggtgcacaaactatccgatgccatactccagaagattattgacagtcgaaaagtagccaaatctcaaggcaaaacacacgaaaacttggttgatattctcctcaaattccataaggataccgggcatgatctcggcttaagcgatgacaatataaaagctgtgctacaggatatgttcattgctggaatcgaaacatcatcgacaactacagattgggccatggcagaaatgatgaaacatccaagccttcttaagaaggcacaagatgaggtgagacagatttttctcgacaagggatttgtcgatgagtccgacttcgatgagctaaagtacctaaacttagtgatcaaagaaactttccgaatccacccgccggggcctttaattctcagagaaaacaaagagacctgtgagatcaatggatacacaatacctgcaaaaacaagggttgtggtgaatgcatgggccattggaagagatcccaaaatctggaaagatgccgacagctttataccagagagatttctcgataacaatatttcagccgattacactggaaagaatttcgattacatcccttttggctctggtcgaaggatttgccctggaatgtcattcggcatagctaacgtcgaattcccattggcaatgttactctaccacttcgattgggttttgccacagggaatcaaacctgaagatgtggacatggctgaatcctttggccttacagcagtaagaaaaactcctcttactgtgattcccaaattgaaaaaccctttgcctgtaaagta

SEQ ID NO: 5 - DNA, 1359 bp, synthetic construct
  source 1..1359: mol_type = other DNA
caccacgacctccggcgcttggcgcgcaaacatggtcctctcatgctgctgcactttggcaccgtgcctacgctcatcgtgtcatccgtggagggcgccaaggaaataatgaaaactcatgatcttgcatttgcagaccggcccgagtcttgggttactaagaggctgctctacgatcacaaggatgtgtcggttgcgccgtatggcgaatattggagacagttgaagagtatatgtgtgctccagcttctcagcaataaaagagtccaatctttccattcgatccgggaagaagaaatgtcccttttgatggaaaaaattaggggctcgtcacgcgcgttgaatttgagtgagatgttcactaaagtcactaatgatatggtttgtagatcggcgtttggtgtgaaatacggcgagggagaaaaggggaagaaattcatggcgcttttgacggagtttttggagttgttagggaccatttacatcggaaattttgtgccgtggctttcgtggattactcgtcttaccgggttcgatgcaagagtggataaggttgcaaaagagctcgacatattgatggaggatgtgatcgaagaaaggattagaagaaatcttgaagataaggctgagaatgaacacaagtatggacaaaattttgtagatattttggtagatatttacaagaacaattctgcaggcatctccatggacagagatagtgtgaaagcaatacttttggatgtttttgcagctgggacggacaccacatcgactgttctagaatgggcgatgagtgaactccttcgacaccccgaggtgatgaagaaactgcaaaatgaagtgaggggagtaatgaaagacaagggcaagataagcgatgaagacttagaaaaaatgcaatatctaaaagccgtgatcaaagaaactcttcgcctgcacactccgatcccagctctagttcctcggatggcaagaaaagatgtcaaggttatgggatacgatgtatcagccgggacaatggtgatgattaatgcttgggccataggcagagaccccggcatttgggatgagcctgaaaagttcaaccctaaccggtttctaaattcatcgatagatttcaagggccaggatttcgagctgatcccttttggggccgggcggcgaggctgcccggggatttcatttgctatggctaccaatgagtttgtgttagcaaatattgtgcataacttcaagtggaaattggctgatgatgagaaagaattagacatgagtgaacgccccggtgtcgccattcgtagggctgttcctcttcttgccgtggcatcaaagttctatactgagggtgtgttcgtttga

SEQ ID NO: 6 - DNA, 1338 bp, synthetic construct
  source 1..1338: mol_type = other DNA
catcatattttagccgacttggcgtcgaaatatggggcgatgatgcacttgcggctaggtgaaatcagcacggttgttatatcgtcgccggaggcagcaaaagaggtcatgaagacatatgacatcaacttcgctaataggccatctttctatgcatcgaagattataacttatgacaactccgacataggattctccaatggagaataccggaaaaatttgcgaaaagtaagcaaaatggagctgctgagtgcgaagcgcgtcaaatcttttcgtcccttaagggaagaagtgttttcggatatgtgcagttggatagcttcaaaagaaggctcgtcgataaacttgactgagaaagtttccttggcgacttttgatgtggctttgcgggcatcgcttggcggaaagattgatgaacacacagcaatggtagatgtaactaaggagtctttcgatttactcgcaggattttatgccggcgatttgtttccctccatcagtttgttgcaatcaatcgacggatttagaggtcgtgtggagaaggtgcacaaactatccgatgccatactccagaagattattgacagtcgaaaagtagccaaatctcaaggcaaaacacacgaaaacttggttgatattctcctcaaattccataaggataccgggcatgatctcggcttaagcgatgacaatataaaagctgtgctacaggatatgttcattgctggaatcgaaacatcatcgacaactacagattgggccatggcagaaatgatgaaacatccaagccttcttaagaaggcacaagatgaggtgagacagatttttctcgacaagggatttgtcgatgagtccgacttcgatgagctaaagtacctaaacttagtgatcaaagaaactttccgaatccacccgccggggcctttaattctcagagaaaacaaagagacctgtgagatcaatggatacacaatacctgcaaaaacaagggttgtggtgaatgcatgggccattggaagagatcccaaaatctggaaagatgccgacagctttataccagagagatttctcgataacaatatttcagccgattacactggaaagaatttcgattacatcccttttggctctggtcgaaggatttgccctggaatgtcattcggcatagctaacgtcgaattcccattggcaatgttactctaccacttcgattgggttttgccacagggaatcaaacctgaagatgtggacatggctgaatcctttggccttacagcagtaagaaaaactcctcttactgtgattcccaaattgaaaaaccctttgcctgtaaagtaa

SEQ ID NO: 7 - DNA, 2070 bp, synthetic construct
  source 1..2070: mol_type = other DNA
atggattcgcggctggagctgttgagatccgtcgagcaagcccttggggtcaagctcggggacgagactgtggtgttggtgcttaccacctcagtagccgtgattttggggctgctcgtctttctttggaagagatcgggcgataatagaagcaaggatgccaggcaggtcgtcgttcccaagccggtctcgctcaaggacgacgacgaggacgaatatgtggggccagacaagaccaagctcaccatcttcttcgggacgcagaccggcaccgccgagggattcgctaaggcattagccgaagagatcaaggcaagatatgataaggcggttgttaaagttgtggacatggatgattatgctcaagatgacgaccaatacgaggaaaagctgaagaaagagactctagcatttttcatggtcgctacttatggagatggtgagccaactgataatgctgctagattttataaatggttcactgagggggaagaaagagaaccctggcttcagcagctttcctatggaatatttggtttgggtaatcgccaatatgaacattttaacaagattggaaaggtcattgatgagaagcttagtgagcaaggtgcaaaacgtctggtacaactaggtctaggagatgatgatcaatgcatcgaagatgatttcactgcctggaaagagcaactatggcctgaattagatcaaattctgaggggtgaagaaagtttggactctgtctctaccccttatacagctgcaataccagaatatagagttgtgatccatgatgctgctattccatcttatgatgacaatcatgcatttgttgctaatggggatgcttcatatgatcttcatcacccttgcagagtcaatgttgctgttcagagagaacttcacacccctgaatctgaccgttcatgtatacatctggagtttgacatatctggaactggtatagtatatgagactggagatcatgttggtgtttttgctgaaaattgcgatgaaactgtcgaagaagcatctaaactattgggacagcctttggatttactattttccatccactctgacaaggatgatggctctccaattggaggttcattaccacctccatttcctggcccctgcacacttcgttctgcactggcacaccacgctgacctgttgaatccacctcgaaaggctgctttgactgcattagccgcacatgcttctgaaaccaatgaagctgaaaagcttaaattcttggcatcccctcagggaaaggatgagtatgcacaatgggttgttggcagtcagagaagtctccttgaggtaatggctgagtttccttcttcaaaacctccacttggggtattttttgctgcaattgctccacgtctgcagccacgctactattctatatcttcatctccaaggtttgccccaactcgtgttcatgtgacatgtgctctagtttatggtccaagtccgactggccgaattcataaaggagtgtgttcgacctggatgaagcatgcagttcccttggagaaaagcagtagctgtagctgggctcctatttttatcaggccatcgaatttcaaattgcctgctgattcttctattccaattgtcatggtgggtcctggaacagggttggcaccttttagaggatttttacaggaaagaatggctctgaaaaatgacggcgctcaacttgggcctgctctatttttttttggttgtaggaaccgccgtcaggattttatttatgaaaatgagctgaatgattttgtggatcaaggagtaatatccgagctgattgttgcattctcaagggagggacaacaaaaggaatatgttcaacataagatgctggaaaaggcagatcaagtttggagtttgatctctcagaaaggatatctctatgtgtgtggagatgcaaaggggatggcgagagatgttcatcgtacattgcacaccatcgtccaaacgcaggaaaatgtcgattcatcaaaggcagagtctatagttaagaaacttcagatggatggccgatacctgagggatgtttggtga

SEQ ID NO: 8 - DNA, 1170 bp, synthetic construct
  source 1..1170: mol_type = other DNA
ccgacgtttctccaaaattcatttcaattttttctatcccaatttcacaaaacaattccgacaatgggatcaatgaatctcgtagacacctgcgccgccaattccgccgccattttccggccggcgccgccaattcttcggagcgccttcgtcggcgccgccagactgccgccgtgtccaatccgactgaaaaaaacgagcattgcgcattccggcggcgtggcggcgattctgacggaggaggacgtgaaactcgccgccgggaaggaggggcggcgggaattcgacttcggccggtacgtggcggagaaggcggagttcgttaacggagctctcgacggcgccgtcgtgatgaggcagccggcggtgatccacgaggcgatgaggtactcgttgctcgccggcgggaagcggatccggccgatgctgtgcatcgccgcctgtgagatcgtcggcggggacccgtccgccgccgtgcccgccgcctgcgcggcggagatgatccacacgatgtcgctgatccacgacgacctgccgtgtatggacaacgacgacctccgccgcggcaagccgaccagtcacaagatgttcggcgagaacgttgctgtccttgcaggtgattctttattagcttttgcattcgaattcatagcgacggggactaaggacgtggcgccggagaggatcatagcatgtgtcgacgaattagcaaaggcggtcgggggcgacggtttagtggcggggcaagtggcagacataaaactcaccggtaataacgcggacgtgggactagacatgttagaattcatacatatccacaagaccgcggtgctactggaggcgtcggtggtaatgggggcaattcttgggggcggaagcccccagcaagtggataagctacggatctttgcgcaaaagatcgggttgctttttcaagtggtggacgacatactagatgtgacgatgtcgtccgaagaattgggaaaaacggcggggaaggacttggccactgacaagacgacgtatccgaagcttttgggagtcgagggggcccgagaatttgcgaataagttgtgcgaggaggcgaaggaacagctggcggagttcgattcggacaaggcggcgccgctggcggcgctggcggagtacattggtcggaggcagaattga

SEQ ID NO: 9 - DNA, 2499 bp, synthetic construct
  source 1..2499: mol_type = other DNA
atgcctttgttttccctcctcggatcatccccctccccgccgccccagctctatattccggcagcatctccttttcctcgcacctccgttgttgcaggactattttctgtttggagaaatacaacaaacactggctatccccttctgcaatgtggtgcagtttccagagctcctgctgaaggagaatatatagatgtgattctgagtggtttccctgtcagaaagtggccatcgaatattgctacagagggtgacacacaaaaggataagtataaaggcgaggaagtgagatcaaatgagaggagggatcggatggttgagatgatcaagtcaatgctgagatcaatggacgatggagagataagcatatctccttatgacactgcgtgggtggcgttggtggaggatattggaggcaaaggacagcctcagtttcccaccagcatcgaatggatttcaaacaaccaactggaggacggttcgtggggcgacagtgctgcctattcagctcacgacaggatactcaacacattggcttgtgttgtcgcattgagatcctggaaaatgcaccaggacaaaactgataaaggggttacatttatcagaaagaacattcacaggctagacgaagaaaaagaagagcacatgcccataggtttcgaagtggcactgccatcccttattgaaacagccaaaaagttagggatagatgtgcccaatgattccccagccttgcaaaaaatttacgctagaagagaattaaagcttacaaggataccaagggacattatgcacaaggtgcccacaacattactgcacagcttagagggaatgtcatcagggcttgagtggcaagagctgttgaaattacagtgtcctgacggctcatttctcttttctccatcctccactgcctttgcattacaacaaactaaggatgagaattgtctcaaatatttactcaagcatgttagaaagttcaatgggggagttcctaatgtgtatccggtggacttatttgagcgcctatgggctgtggatcggctacaaaggctggggatatcaaggtattttcaacctgagattgaagagtgccttggttatgtacacagatactggacagacaaggggatttgttgggcaagaaattctcaagttcaggccgttgatgacactgccatgggattcaggcttctgaggttgcatggctaccgggtttcggcagatgttttcaagaactttaaacaggacgaagagttcttctgcttcgcggggcagtcaaatcaggcagtgactggaatgtataacttgtacagggcttctcaggtgatgtttccaggggaagttatacttgcagaagcccgcaagttctcccacaagttcttgcaagagaaaagggctaacaatgaactacttgataaatggattataaccaaagaccttccaggagaggtgggatttgcactggatattccatggtatgccagtttacctagaattgagacaagatttttcttggaacaatatggtggtggtgatgatgtttggattgggaagaccttgtacagaatgccctacattgacaacagggcctaccttgatctggcaaagttagattacaataattgccaggcattgcatcagagtgagtggaaaagctttcaaaaatggtacagaagttgcagacttgaggagtttgggttgtcggagacgacccttgttcaaacttattacatcgcagcagcaagcatatttgagccagaaagattgtatgagcggcttgcatgggctaaaactgcgattctaatggagactgtcatgcggcattcggaccaaaagaaactctccaagcaacaaaagcacgcattagtcaatgaattcaaacattgtgcactgcttggagaaaggtacaaaacacgaaacaacctgatagggaccctggtcagaagtgtgaatgaactctcattggacgcaccattggcgcgatacgcagacatccaatcacacttgcatcgggcgtggcaaaagtggctaagctcatgggaggaaggggacacgggtgaaggcgatgcagagctgctagtgtgcactctaaatctgtgtggtggaggccgcagcagtcacagtcgatggtccgacaaattattgttgtcgtatcctccgtatcagccccttgtgcagatcgccagcagagtctgtcaccatctccgcctgtctccaacccgaaaggaggaggcaaggatgtgcgagcagaagctgcaaagtggcgtgagtggtggtgacatagaagggagcatgcaagagctggtgagattagcgctggccaagagccacagcgaattggagtttcgtgtgaagcaaaacttcctccttgtagccaggagttattactacaccgcatattgtaaccctgctactatcaatctccacatagccaaagtcctctttgagactgtcgtactagaatgctgtgatgaccatacccgccgttacttctga

SEQ ID NO: 10 - DNA, 725 bp, synthetic construct
  source 1..725: mol_type = other DNA
tttgccagcttactatccttcttgaaaatatgcactctatatcttttagttcttaattgcaacacatagatttgctgtataacgaattttatgctattttttaaatttggagttcagtgataaaagtgtcacagcgaatttcctcacatgtagggaccgaattgtttacaagttctctgtaccaccatggagacatcaaaaattgaaaatctatggaaagatatggacggtagcaacaagaatatagcacgagccgcggagttcatttcgttacttttgatatcactcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 11 - DNA, 700 bp, synthetic construct
  source 1..700: mol_type = other DNA
gtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaaca

SEQ ID NO: 12 - DNA, 401 bp, synthetic construct
  source 1..401: mol_type = other DNA
aaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctat

SEQ ID NO: 13 - DNA, 39 bp, synthetic construct
  source 1..39: mol_type = other DNA
ctgcccagccggcgatggccaccaagaacttgcccccgt

SEQ ID NO: 14 - DNA, 49 bp, synthetic construct
  source 1..49: mol_type = other DNA
cagtggtggtggtggtggtgaacgaacacaccctcagtatagaactttg

SEQ ID NO: 15 - DNA, 39 bp, synthetic construct
  source 1..39: mol_type = other DNA
taagaaggagatataccatgaaacgtccccggagttccg

SEQ ID NO: 16 - DNA, 50 bp, synthetic construct
  source 1..50: mol_type = other DNA
cagtggtggtggtggtggtgctttacaggcaaagggtttttcaatttggg

SEQ ID NO: 17 - DNA, 37 bp, synthetic construct
  source 1..37: mol_type = other DNA
caccagctgagctcattgcctcaccacgacctccggc

SEQ ID NO: 18 - DNA, 49 bp, synthetic construct; identical to SEQ ID NO: 14
  source 1..49: mol_type = other DNA

SEQ ID NO: 19 - DNA, 44 bp, synthetic construct
  source 1..44: mol_type = other DNA
gtcagctcccgaccaccgcatcatattttagccgacttggcgtc

SEQ ID NO: 20 - DNA, 50 bp, synthetic construct; identical to SEQ ID NO: 16
  source 1..50: mol_type = other DNA

SEQ ID NO: 21 - DNA, 58 bp, synthetic construct
  source 1..58: mol_type = other DNA
gaatttgacaatgttaagctttgttaaacagcttggcgtaatcatggtcatagctgtt

SEQ ID NO: 22 - DNA, 42 bp, synthetic construct
  source 1..42: mol_type = other DNA
ggtagttccacgcggccgatccgagttctaattcactggccg

SEQ ID NO: 23 - DNA, 40 bp, synthetic construct
  source 1..40: mol_type = other DNA
ggccagtgaattagaactcggatcggccgcgtggaactac

SEQ ID NO: 24 - DNA, 48 bp, synthetic construct
  source 1..48: mol_type = other DNA
aaggatagtaagctggcaaatgttgattgtctaactgcgttcttttgt

SEQ ID NO: 25 - DNA, 56 bp, synthetic construct
  source 1..56: mol_type = other DNA
aagaacgcagttagacaatcaacatttgccagcttactatccttcttgaaaatatg

SEQ ID NO: 26 - DNA, 59 bp, synthetic construct
  source 1..59: mol_type = other DNA
aattttggagaaacgtcggttttgagggaatattcaactgtttttttttatcatgttga

SEQ ID NO: 27 - DNA, 54 bp, synthetic construct
  source 1..54: mol_type = other DNA
agttgaatattccctcaaaaccgacgtttctccaaaattcatttcaattttttc

SEQ ID NO: 28 - DNA, 45 bp, synthetic construct
  source 1..45: mol_type = other DNA
tagaaacattttgaagctattcaattctgcctccgaccaatgtac

SEQ ID NO: 29 - DNA, 58 bp, synthetic construct
  source 1..58: mol_type = other DNA
attggtcggaggcagaattgaatagcttcaaaatgtttctactccttttttactcttc

SEQ ID NO: 30 - DNA, 56 bp, synthetic construct
  source 1..56: mol_type = other DNA
aggagggaaaacaaaggcatcttagattagattgctatgctttctttctaatgagc

SEQ ID NO: 31 - DNA, 42 bp, synthetic construct
  source 1..42: mol_type = other DNA
gcatagcaatctaatctaagatgcctttgttttccctcctcg

SEQ ID NO: 32 - DNA, 46 bp, synthetic construct
  source 1..46: mol_type = other DNA
gcataaatttttagttaaagggtcagaagtaacggcgggtatggtc

SEQ ID NO: 33 - DNA, 53 bp, synthetic construct
  source 1..53: mol_type = other DNA
catacccgccgttacttctgaccctttaactaaaaatttatgcatttggctcc

SEQ ID NO: 34 - DNA, 58 bp, synthetic construct
  source 1..58: mol_type = other DNA
tatgaccatgattacgccaagctgtttaacaaagcttaacattgtcaaattcttcagg

SEQ ID NO: 35 - DNA, 58 bp, synthetic construct
  source 1..58: mol_type = other DNA
tgttaatacttctaactatagttttagagctagaaatagcaagttaaaataaggctag

SEQ ID NO: 36 - DNA, 51 bp, synthetic construct
  source 1..51: mol_type = other DNA
tatagttagaagtattaacagatcatttatctttcactgcggagaagtttc

SEQ ID NO: 37 - DNA, 39 bp, synthetic construct
  source 1..39: mol_type = other DNA
gtaaagggggsggggsggggsatggattcgcggctggag

SEQ ID NO: 38 - DNA, 44 bp, synthetic construct
  source 1..44: mol_type = other DNA
gaatgtaagcgtgacataactcaccaaacatccctcaggtatcg

SEQ ID NO: 39 - DNA, 47 bp, synthetic construct
  source 1..47: mol_type = other DNA
accaaacctctggcgaagaatttgccagcttactatccttcttgaaa

SEQ ID NO: 40 - DNA, 59 bp, synthetic construct
  source 1..59: mol_type = other DNA
gggggcaagttcttggtcatttttgagggaatattcaactgtttttttttatcatgttg

SEQ ID NO: 41 - DNA, 40 bp, synthetic construct
  source 1..40: mol_type = other DNA
agttgaatattccctcaaaaatgaccaagaacttgccccc

SEQ ID NO: 42 - DNA, 49 bp, synthetic construct
  source 1..49: mol_type = other DNA
cattttgaagctatgagctcaacgaacacaccctcagtatagaactttg

SEQ ID NO: 43 - DNA, 61 bp, synthetic construct
  source 1..61: mol_type = other DNA
caaagttctatactgagggtgtgttcgttgagctcatagcttcaaaatgtttctactcctt

SEQ ID NO: 44 - DNA, 59 bp, synthetic construct
  source 1..59: mol_type = other DNA
gaactccggggacgtttcatactagttctagaaaacttagattagattgctatgctttc

SEQ ID NO: 45 - DNA, 41 bp, synthetic construct
  source 1..41: mol_type = other DNA
ctaagttttctagaactagtatgaaacgtccccggagttcc

SEQ ID NO: 46 - DNA, 50 bp, synthetic construct
  source 1..50: mol_type = other DNA
atccatsccccsccccsccccctttacaggcaaagggtttttcaatttgg

SEQ ID NO: 47 - DNA, 45 bp, synthetic construct
  source 1..45: mol_type = other DNA
acctgagggatgtttggtgagttatgtcacgcttacattcacgcc

SEQ ID NO: 48 - DNA, 41 bp, synthetic construct
  source 1..41: mol_type = other DNA
aaggatagtaagctggcaaattcttcgccagaggtttggtc

SEQ ID NO: 49 - DNA, 10251 bp, synthetic construct
  source 1..10251: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggcatagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaattttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttctagaactagtggatcccccgggaaaaatggacaagaagtactccattgggctcgatatcggcacaaacagcgtcggttgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtgatctcttctcgagtcatgtaattagttatgtcacgcttacattcacgccctccccccacatccgctctaaccgaaaaggaaggagttagacaacctgaagtctaggtccctatttatttttttatagttatgttagtattaagaacgttatttatatttcaaatttttcttttttttctgtacagacgcgtgtacgcatgtaacattatactgaaaaccttgcttgagaaggttttgggacgctcgaaggctttaatttgcctcgaggtcgacggtatcgataagcttgatatcgaattcctgcagcccgggggatccactagttctttgaaaagataatgtatgattatgctttcactcatatttatacagaaacttgatgttttctttcgagtatatacaaggtgattacatgtacgtttgaagtacaactctagattttgtagtgccctcttgggctagcggtaaaggtgcgcattttttcacaccctacaatgttctgttcaaaagattttggtcaaacgctgtagaagtgaaagttggtgcgcatgtttcggcgttcgaaacttctccgcagtgaaagataaatgatcgcctttatggagaaaaatgggttttagagctagaaatagcaagttaaaataaggctagtccgttatcaacttgaaaaagtggcaccgagtcggtggtgctttttttgttttttatgtcgagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgaacgaagcatctgtgcttcattttgtagaacaaaaatgcaacgcgagagcgctaatttttcaaacaaagaatctgagctgcatttttacagaacagaaatgcaacgcgaaagcgctattttaccaacgaagaatctgtgcttcatttttgtaaaacaaaaatgcaacgcgagagcgctaatttttcaaacaaagaatctgagctgcatttttacagaacagaaatgcaacgcgagagcgctattttaccaacaaagaatctatacttcttttttgttctacaaaaatgcatcccgagagcgctatttttctaacaaagcatcttagattactttttttctcctttgtgcgctctataatgcagtctcttgataactttttgcactgtaggtccgttaaggttagaagaaggctactttggtgtctattttctcttccataaaaaaagcctgactccacttcccgcgtttactgattactagcgaagctgcgggtgcattttttcaagataaaggcatccccgattatattctataccgatgtggattgcgcatactttgtgaacagaaagtgatagcgttgatgattcttcattggtcagaaaattatgaacggtttcttctattttgtctctatatactacgtataggaaatgtttacattttcgtattgttttcgattcactctatgaatagttcttactacaatttttttgtctaaagagtaatactagagataaacataaaaaatgtagaggtcgagtttagatgcaagttcaaggagcgaaaggtggatgggtaggttatatagggatatagcacagagatatatagcaaagagatacttttgagcaatgtttgtggaagcggtattcgcaatattttagtagctcgttacagtccggtgcgtttttggttttttgaaagtgcgtcttcagagcgcttttggttttcaaaagcgctctgaagttcctatactttctagagaataggaacttcggaataggaacttcaaagcgtttccgaaaacgagcgcttccgaaaatgcaacgcgagctgcgcacatacagctcactgttcacgtcgcacctatatctgcgtgttgcctgtatatatatatacatgagaagaacggcatagtgcgtgtttatgcttaaatgcgtacttatatgcgtctatttatgtaggatgaaaggtagtctagtacctcctgtgatattatcccattccatgcggggtatcgtatgcttccttcagcactaccctttagctgttctatatgctgccactcctcaattggattagtctcatccttcaatgctatcatttcctttgatattggatcatactaagaaaccattattatcatgacattaacctataaaaataggcgtatcacgaggccctttcgtc

(19) United States
(12) Patent Application Publication
(10) Pub. No.: US 2024/0117387 A1
(43) Pub. Date: Apr. 11, 2024

(54) P450 Cytochrome Enzyme for Andrographolide Synthesis and Its Application

(71) Applicant: Jiangnan University, Wuxi (CN)

(72) Inventors: Jingwen Zhou, Wuxi (CN); Shan Li, Wuxi (CN); Song Gao, Wuxi (CN); Sha Xu, Wuxi (CN); Weizhu Zeng, Wuxi (CN); Shiqin Yu, Wuxi (CN)

(21) Appl. No.: 18/541,098

(22) Filed: Dec. 15, 2023

(30) Foreign Application Priority Data
Dec. 24, 2022 (CN) 2022116693851

(51) Int. Cl. C12P 7/02; C12N 9/10; C12N 9/88; C12N 15/81; C12P 5/00

(52) U.S. Cl. CPC C12P 7/02; C12N 9/1085; C12N 9/88; C12N 15/81; C12P 5/002; C12Y 205/01; C12Y 402/03028; C12N 2800/102

(57) ABSTRACT

The present disclosure provides a P450 cytochrome enzyme for andrographolide synthesis and its application, belonging to the field of bioengineering. The present disclosure uses Saccharomyces cerevisiae CEN.PK2-1D as a host, and implements knockout of ROX1 and GAL80 genes on the genome, and integrative expression of GGPP synthase encoding gene and CPS diterpene synthase encoding gene at ROX1 site; and implements free expression of ApCPR and CYP71A8 and CYP71D10 both with truncated signal peptides, successfully constructing recombinant S. cerevisiae, and achieving de novo synthesis of 3,15,19-Trihydroxy-8(17),13-ent-labdadiene-16-oic acid. Compared with the blank, a response value of a product peak reaches 1.9*106, and this strategy provides necessary reference for analyzing biosynthetic pathway of andrographolide and using metabolic engineering to synthesize andrographolide and related derivatives thereof.

### REFERENCE TO SEQUENCE LISTING

The instant application contains a Sequence Listing in XML format as a file named “YGHY-2023-50-SEQ.xml”, created on Dec. 13, 2023, of 69.7 kB in size, and which is hereby incorporated by reference in its entirety.

### TECHNICAL FIELD

The present disclosure relates to a P450 cytochrome enzyme for andrographolide synthesis and its application, belonging to the field of applied microbiology and enzyme engineering.

### BACKGROUND

Cytochrome P450 (P450) belongs to the heme oxygenase superfamily, named after the maximum absorption peak near 450 nm when the reduced heme oxygenase forms a complex with CO. According to the cellular localization of P450 enzymes and the composition of redox partner proteins (NADPH-cytochrome P450 reductase, CPR), they can be divided into 5 classes. Class I requires additional electron transport proteins to transfer electrons NAD(P)H to P450 enzymes active center to complete the entire catalytic process.

ent-Copalol and 3,15,19-Trihydroxy-8(17),13-ent-labdadiene-16-oic acid are diterpene compounds containing four isoprenes, which are present in herb Andrographis paniculata and are important intermediate compounds for synthesis of andrographolide. Andrographolide has heat-clearing, detoxifying, swelling reducing and analgesic effects, and is known as a “natural antibiotic drug”. However, there are currently no reports on catalytic reactions of the P450 enzymes and pathway substances 3,15,19-Trihydroxy-8(17),13-ent-labdadiene-16-oic acid for the synthesis of andrographolide. Therefore, studying the activity and function of CYP450 enzymes in the synthesis process is of great significance for understanding the entire synthesis pathway of andrographolide.

CYP450 enzymes are membrane proteins, and their formation requires a series of post-translational modifications. Eukaryotic expression hosts have a relatively complete post-translational modification system including endoplasmic reticulum, Golgi apparatus and other inner membrane structures, making the expressed heterologous protein closer to their natural conformation. Eukaryotic microorganisms are usually considered suitable systems for heterologous expression of CYP450 enzymes. In addition, endogenous CPR of eukaryotic microorganisms can mediate effective transfer of electrons to the CYP450 enzymes, helping the CYP450 enzymes to exert their catalytic function. The natural advantages of eukaryotic microorganisms are applied in catalytic synthesis of 3,15,19-Trihydroxy-8(17),13-ent-labdadiene-16-oic acid (FIG. 1). According to the existing research, there is currently no eukaryotic microbial recombinant strains for de novo synthesis of 3,15,19-Trihydroxy-8(17),13-ent-labdadiene-16-oic acid.

### SUMMARY

In the present disclosure, CYP71A8 and CYP71D10 derived from Andrographis paniculata belong to the Class I CYP71 family and require electron transport protein to catalyze their activity, but their specific biological functions have not been reported. Therefore, the present disclosure is the first to perform heterologous expression of CYP71A8 and CYP71D10 and provide their catalytic functions.

A first objective of the present disclosure is to provide application of CYP450 enzymes derived from Andrographis paniculata in catalytic synthesis of 3,15,19-Trihydroxy-8(17),13-ent-labdadiene-16-oic acid.

In one embodiment, the CYP450 enzymes derived from Andrographis paniculata are CYP71A8 and CYP71D10.

In one embodiment, a nucleotide sequence of the CYP71A8 is shown in SEQ ID NO:1; and a nucleotide sequence of the CYP71D10 is shown in SEQ ID NO:2.

In one embodiment, the CYP71A8 has N-terminal truncated by 32 amino acids, and the CYP71D10 has N-terminal truncated by 28 amino acids.

In one embodiment, a nucleotide sequence of the CYP71A8 with N-terminal truncated by 32 amino acids is shown in SEQ ID NO:3, and a nucleotide sequence of the CYP71D10 with N-terminal truncated by 28 amino acids is shown in SEQ ID NO:4.

A second objective of the present disclosure is to provide a recombinant Saccharomyces cerevisiae, including knockout of ROX1 and GAL80 genes on the genome; integrative expression of a GGPP synthase encoding gene and CPS diterpene synthase encoding gene; and free expression of CYP71A8 encoding gene CYP71A8t with N-terminal truncated by 32 amino acids, CYP71A10 encoding gene CYP71D10t with N-terminal truncated by 28 amino acids, and CPR encoding gene ApCPR.

In one embodiment, after the knockout of ROX1 site, the GGPP synthase encoding gene and the CPS diterpene synthase encoding gene are integrated at the ROX1 site.

In one embodiment, a nucleotide sequence of the CYP71A8 with N-terminal truncated by 32 amino acids is shown in SEQ ID NO:3, and a nucleotide sequence of the CYP71D10 with N-terminal truncated by 28 amino acids is shown in SEQ ID NO:4.

In one embodiment, a nucleotide sequence of the ApCPR is shown in SEQ ID NO:7.

In one embodiment, a nucleotide sequence of the GGPP synthase is shown in SEQ ID NO:8.

In one embodiment, a nucleotide sequence of the CPS diterpene synthase is shown in SEQ ID NO:9.

In one embodiment, S. cerevisiae CEN.PK2-1D is used as a starting strain.

In one embodiment, promoter P_{PGK1} is used for starting expression of the CPR encoding gene.

In one embodiment, promoter P_{TEF1} is used for starting expression of the CYP71A8t.

In one embodiment, promoter P_{GAL7} is used for starting expression of the CYP71D10t.

In one embodiment, a nucleotide sequence of the promoter P_{GAL7} is shown in SEQ ID NO:10, a nucleotide sequence of the promoter P_{PGK1} is shown in SEQ ID NO:11, and a nucleotide sequence of the promoter P_{TEF1} is shown in SEQ ID NO:12.

In one embodiment, pY26 series vectors or pET series vectors are used as expression vectors.

In one embodiment, the pET series vectors include a pET22b(+) expression vector or a pET28a(+) expression vector.

A third objective of the present disclosure is to provide a whole-cell catalyst, which contains the above-mentioned recombinant S. cerevisiae.

A fourth objective of the present disclosure is to provide a method for synthesizing 3,15,19-Trihydroxy-8(17),13-ent-labdadiene-16-oic acid, using the above-mentioned recombinant S. cerevisiae or the above-mentioned whole-cell catalyst as a fermentation strain for fermentation to produce 3,15,19-Trihydroxy-8(17),13-ent-labdadiene-16-oic acid.

In one embodiment, the method includes fermentation to produce in a culture medium using glucose as a carbon source.

In one embodiment, the method includes inoculating seed liquid of the above-mentioned recombinant S. cerevisiae or the above-mentioned whole-cell catalyst into the culture medium, and fermenting at 28-32° C. and 200-230 rpm for 90-160 h.

In one embodiment, the culture medium includes 10-30 g/L peptone, 5-15 g/L yeast powder, and 10-30 g/L glucose.

In one embodiment, a method for preparing the seed liquid includes inoculating the above-mentioned recombinant S. cerevisiae or the above-mentioned whole-cell catalyst into a seed culture medium, and fermenting at 28-32° C. and 200-230 rpm for 12-20 h.

The present disclosure further provides application of the CYP450 enzymes derived from Andrographis paniculata, or the above-mentioned recombinant S. cerevisiae, or the above-mentioned whole-cell catalyst, or the above-mentioned method in catalytic synthesis of andrographolide or products containing andrographolide.

In one embodiment, the CYP450 enzymes derived from Andrographis paniculata are CYP71A8 and CYP71D10.

In one embodiment, a nucleotide sequence of the CYP71A8 is shown in SEQ ID NO:1; and a nucleotide sequence of the CYP71D10 is shown in SEQ ID NO:2.

In one embodiment, the CYP71A8 has N-terminal truncated by 32 amino acids, and the CYP71D10 has N-terminal truncated by 28 amino acids.

In one embodiment, a nucleotide sequence of the CYP71A8 with N-terminal truncated by 32 amino acids is shown in SEQ ID NO:3, and a nucleotide sequence of the CYP71D10 with N-terminal truncated by 28 amino acids is shown in SEQ ID NO:4.

The present disclosure further provides application of the above-mentioned recombinant S. cerevisiae, or the above-mentioned whole-cell catalyst, or the above-mentioned method in catalytic synthesis of 3,15,19-Trihydroxy-8(17),13-ent-labdadiene-16-oic acid or products containing 3,15,19-Trihydroxy-8(17),13-ent-labdadiene-16-oic acid.

The Present Disclosure has the Following Beneficial Effects:

1. The present disclosure uses BL21 (DE3) as a host to express recombinant plasmids pET22b(+)-CYP71A8t and pET28a(+)-CYP71D10t, uses obtained recombinant Escherichia coli to successfully implement heterologous expression of CYP7A8 and CYP71D10 with truncated signal peptides, and finds that the protein loses expression activity when CYP71A8 is truncated to 57 amino acids, and the protein loses expression activity when CYP71D10 is truncated to 59 amino acids.

2. The present disclosure uses S. cerevisiae CEN.PK2-1D as a host, and implements knockout of the ROX1 and GAL80 genes on the genome, and integrative expression of the GGPP synthase encoding gene and the CPS diterpene synthase encoding gene at the ROX1 site; and implements free expression of CYP71A8 and CYP71D10 and ApCPR, successfully constructing recombinant S. cerevisiae CW1006/pY26-P_{TEF1}-CYP71A8t-P_{GAL7}-CYP71D10t, and achieving microbial fermentation to synthesize 3,15,19-Trihydroxy-8(17),13-ent-labdadiene-16-oic acid.

Since there are currently no product standards on the market, LCMS-IT-TOF ion fragments are used for judging product synthesis. Compared with a blank control group carrying pY26-GPD-TEF empty vector plasmid, a response value of a product peak is 1.9*10⁶. The present disclosure has achieved de novo synthesis of 3,15,19-Trihydroxy-8(17),13-ent-labdadiene-16-oic acid for the first time. This strategy provides necessary references for analyzing biosynthesis pathway of andrographolide and using metabolic engineering to synthesize andrographolide and related derivatives thereof.

### BRIEF DESCRIPTION OF FIGURES

FIG. 1: Reaction diagram for catalytic synthesis of 3,15,19-Trihydroxy-8(17),13-ent-labdadiene-16-oic acid by eukaryotic microorganisms.

FIG. 2: Vector map of pRS426-TEF1-Cas9-gRNA-URA3.

FIG. 3: Plasmid map of pET22b(+)-P_{T7}-CYP71A8t and pET28a(+)-P_{T7}-CYP71D10t.

FIG. 4: SDS-PAGE diagram of BL21 (DE3) expressing CYP71A8t and CYP71D10t; 1: pET22b empty vector; 2: pET22b-CYP71A8t; M: Marker; 3: pET28a empty vector; and 4: pET28a-CYP71D10t.

FIG. 5: Plasmid map of pY26-P_{TEF1}-CYP71A8t-P_{GAL7}-CYP71D10ts.

FIG. 6: LCMS-IT-TOF ion chromatogram of recombinant S. cerevisiae catalyzing ent-Copalol to synthesize 3,15,19-Trihydroxy-8(17),13-ent-labdadiene-16-oic acid.

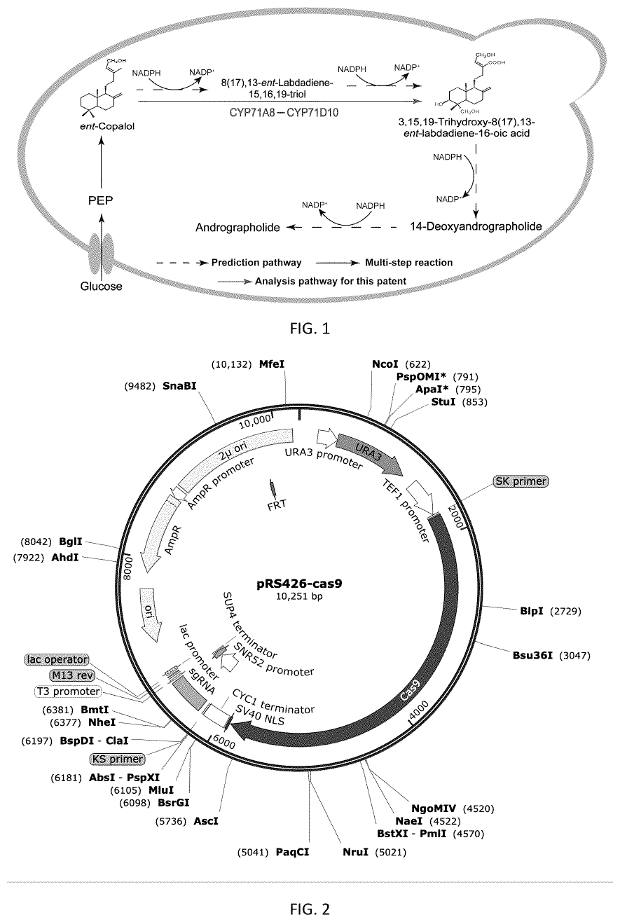

### DETAILED DESCRIPTION

(I) Culture medium

LB medium: peptone 10 g/L, yeast powder 5 g/L, and sodium chloride 10 g/L. 20 g/L agar powder is added to prepare an LB solid culture medium.

YPD medium: peptone 20 g/L, yeast powder 10 g/L, and glucose 20 g/L. 20 g/L agar powder is added to prepare a YPD solid medium.

TB medium: peptone 12 g/L, yeast powder 24 g/L, dipotassium hydrogen phosphate 12.54 g/L, potassium dihydrogen phosphate 2.31 g/L, and glycerol 5 g/L.

YNB medium: glucose 20 g/L, and 20 mL/LYNB medium (purchased from Sangon Biotech (Shanghai) Co., Ltd.). 20 g/L agar powder is added to prepare a YNB solid medium.

(II) Strains and plasmids

pY26-GDP-TEF, pET22b(+), pET28a(+) and pMD-19 vectors are all commercial plasmids, S. cerevisiae CEN.PK2-1D is a commercial yeast cell, and chassis cell C800 is a publicly available strain, recorded in Promoter-Library-Based Pathway Optimization for Efficient (2S)-Naringenin Production From p-Coumaric Acid in Saccharomyces cerevisiae, Song Gao, Hengrui zhou, Jingwen Zhou, Jian chen. J Agric Food Chem, 2020 Jun. 24.

A pRS426-TEF1-Cas9-gRNA-URA3 vector (SEQ ID NO:49) is synthesized by Jiangsu Genecefe Biotechnology Co., Ltd., and the vector map is shown in FIG. 2.

(III) 3,15,19-Trihydroxy-8(17),13-ent-labdadiene-16-oic acid detection conditions

Measurement is performed using LCMS-IT-TOF. Conditions: Chromatographic column C18 (4.6 mm×250 mm, 5 μm); mobile phase methanol (B)-water (A), gradient elution (0-24 min, 30-60% B; 24-40 min, 60% B; 40-46 min, 60%-30% B; 46-50 min, 30% B); column temperature 35° C.; flow rate 0.5 mL/min; and injection volume 5 μL.

### Example 1 Construction of Recombinant Plasmids pET22b(+)-CYP71A8t and pET28a(+)-CYP71D10t

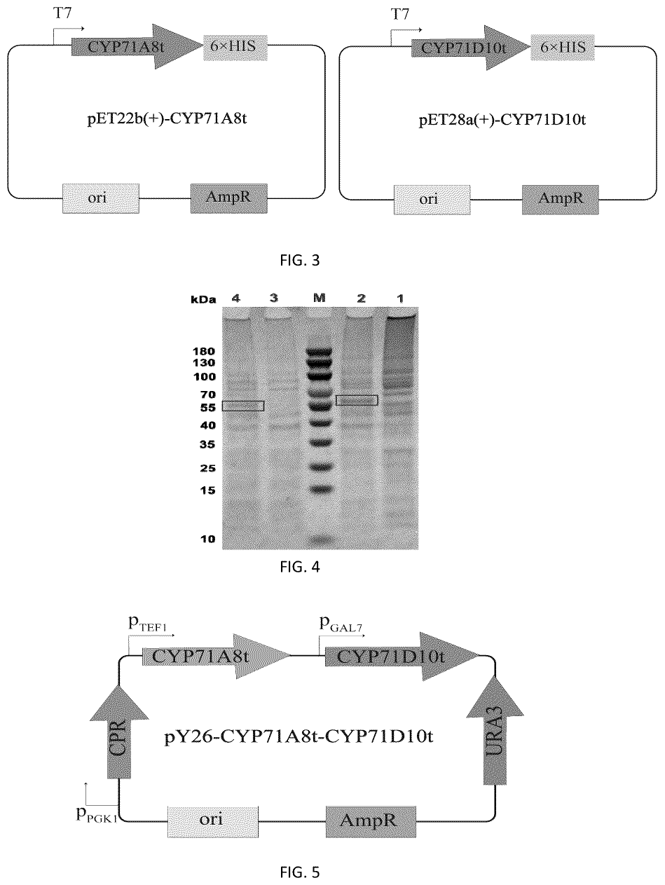

Two original CYP450 enzymes CYP71A8 (a nucleotide sequence as shown in SEQ ID NO:1) and CYP71D10 (a nucleotide sequence as shown in SEQ ID NO:2) derived from Andrographis paniculata were used as templates respectively. PCR amplification was performed using primer pairs 71A8t-F/71A8t-R and 71D10t-F/71D10t-R to obtain target fragments CYP71A8t (a nucleotide sequence as shown in SEQ ID NO:3) and CYP71D10t (a nucleotide sequence as shown in SEQ ID NO:4) both with truncated signal peptides. The amplified CYP71A8t and CYP71D10t were verified by 1.5% agarose gel electrophoresis and then subjected to Gibson assembly with the pET22b(+) and pET28a(+) expression vectors respectively. The assembled plasmids were transferred into E. coli JM109 competent cells, spread on an LB solid medium containing corresponding antibiotics, and cultured at 37° C. overnight. Positive clones were selected and plasmids were extracted. After sequencing was correct, the plasmids were reserved for expression of recombinant E. coli BL21 (DE3) protein. The plasmids were named pET22b(+)-P_{T7}-CYP71A8t and pET28a(+)-P_{T7}-CYP71D10t respectively (plasmid map shown in FIG. 3).

TABLE 1
Primers used for constructing CYP71A8t and CYP71D10t expression vectors

| Primer | Sequence (5′-3′) | |
|---|---|---|
| 71A8t-F | CTGCCCAGCCGGCGATGGCCACCAAGAACTTGCCCCCGT | SEQ ID NO: 13 |
| 71A8t-R | CAGTGGTGGTGGTGGTGGTGAACGAACACACCCTCAGTATA | SEQ ID NO: 14 |
| | GAACTTTG | |
| 71D10t-F | TAAGAAGGAGATATACCATGAAACGTCCCCGGAGTTCCG | SEQ ID NO: 15 |
| 71D10t-R | CAGTGGTGGTGGTGGTGGTGCTTTACAGGCAAAGGGTTTTT | SEQ ID NO: 16 |
| | CAATTTGGG | |

### Example 2 Heterologous Expression of CYP71A8t and CYP71D10t

After the plasmids pET22b(+)-P_{T7}-CYP71A8t and pET28a(+)-P_{T7}-CYP71D10t constructed in Example 1 were respectively transferred into E. coli BL21 competent cells, single clones were selected and inoculated into an LB medium containing 100 mg/L ampicillin and 50 mg/L kanamycin respectively, and cultured at 37° C. and 220 rpm for 16 h to serve as seed liquid. Then the seed liquid in a volume ratio of 1% inoculation amount was respectively transferred to a TB medium with ampicillin or kanamycin (50 mL medium in a 250 mL shaking flask, 50 g/L antibiotics, and 1/1000 volume ratio), and cultured at 37° C. and 220 rpm until OD₆₀₀ of fermentation broth reached 0.6-0.8. A final concentration of 80 mg/L 5-aminolevulinic acid hydrochloride, 10 mg/L FeSO₄ solution and 0.5 mM IPTG were added to induce expression of CYP71A8t and CYP71D10t proteins. After induction at 16° C. for 20 h, the cells were collected by centrifugation at 4° C. and 6000 rpm for 15 min.

After washing the collected cells twice with PBS buffer (pH=7.4), lysis buffer (500 mM potassium phosphate, pH 7.4, 250 mM NaCl, 0.25% sodium cholate, 10% glycerol, 10 mM imidazole, and 10 mM β-mercaptoethanol) was added to resuspend the cells. The cells were sonicated on ice for 3 minutes, and cell lysate was centrifuged at 4° C. and 10000 rpm for 20 min to remove cell debris. Supernatant was collected and subjected to SDS-PAGE protein verification (results shown in FIG. 4).

### Example 3 Expression Identification of Recombinant Plasmids pET22b(+)-CYP71A8t57 and pET28a(+)-CYP71D10t59

Using original sequences of CYP71A8 and CYP71D10 as templates and using primers (Table 2), 57 and 59 amino acids were truncated respectively, and named CYP71A8t57 (a nucleotide sequence as shown in SEQ ID NO:5) and CYP71D10t59 (a nucleotide sequence as shown in SEQ ID NO:6). A method for constructing recombinant plasmids was the same as in Example 1, and the corresponding plasmids were named pET22-CYP71A8t57 and pET28-CYP71D10t59. Induction and identification methods were the same as in Example 2. Supernatant of lysate was collected. SDS-PAGE protein electrophoresis results showed that the protein was not expressed.

TABLE 2
Primers used for constructing CYP71A8t57 and CYP71D10t59 expression vectors

| Primer | Sequence (5′-3′) | |
|---|---|---|
| 71A8t57-F | CACCAGCTGAGCTCATTGCCTCACCACGACCTCCGGC | SEQ ID NO: 17 |
| 71A8t57-R | CAGTGGTGGTGGTGGTGGTGAACGAACACACCCTCAGTATAGAAC | SEQ ID NO: 18 |
| | TTTG | |
| 71D10t59-F | GTCAGCTCCCGACCACCGCATCATATTTTAGCCGACTTGGCGTC | SEQ ID NO: 19 |
| 71D10t59-R | CAGTGGTGGTGGTGGTGGTGCTTTACAGGCAAAGGGTTTTTCAAT | SEQ ID NO: 20 |
| | TTGGG | |

### Example 4 Construction of S. cerevisiae CW1006

Using S. cerevisiae CEN.PK2-1D genome as a template, primer pairs 2UProx1-F/2UProx1-R and 7DOWNrox1-F/7DOWNrox1-R were used to amplify and obtain upstream and downstream homology fragments of ROX1 respectively, primer 3GAL7-F/3GAL7-R was used to amplify and obtain a promoter P_{GAL7} fragment, and primer 5TEF1-F/5TEF1-R was used to amplify and obtain a promoter P_{TEF1} fragment. Using Andrographis paniculata genome as a template, primer 4 ApGGPPS-F/4 ApGGPPS-R was used to amplify and obtain an ApGGPPs fragment, and primer 6ApCPS-F/6ApCPS-R was used to amplify and obtain an ApCPS fragment. Using pMD-19 vector as a template, primer 1GJ-F/1GJ-R was used to amplify and obtain a linearized fragment of vector pMD-19. The above fragments were purified and subjected to Gibson assembly to obtain vector pMD19T-UProx1-P_{GAL7}-ApGGPPs-P_{TEF1}-ApCPS-DOWNrox1. The obtained vector was transformed into E. coli JM109 and sequenced for verification, and positive recombinant vector pMD19T-UProx1-P_{GAL7}-ApGGPPs-P_{TEF1}-ApCPS-DOWNrox1 was obtained. Using the above vector as a template, primer 2UProx1-F/7DOWNrox1-F was used to amplify and obtain a Donar-ROX1 fragment.

Using pRS426-TEF1-Cas9-gRNA-URA3 vector as a template, primer Cas9-F/Cas9-R was used to amplify and obtain linearized vector pRS426-TEF1-Cas9-gRNA-URA3. The obtained vector was transformed into E. coli JM109 and sequenced for verification. Recombinant vector pRS426-20nt with a correct sequence was obtained.

The above Donar-ROX1 fragment and pRS426-20nt were transformed into chassis cell C800, spread on YNB solid plates (containing 50 mg/L Leu, His and Trp), and cultured at 30° C. for 2-3 days. Positive clone strains were selected and passaged multiple times, then pRS426 plasmid was lost (cells can grow on YPD solid plates, but cannot grow on the YNB solid plates containing 50 mg/L Leu, His and Trp), and the CW1006 strain was successfully constructed.

TABLE 3
Primers used for constructing CW1006 strain

| Primer | Sequence (5′-3′) | |
|---|---|---|
| 1GJ-F | gaatttgacaatgttaagctttgttaaacagcttggcgtaatcatggtcatag | SEQ ID NO: 21 |
| | ctgtt | |
| 1GJ-R | ggtagttccacgcggccgatccgagttctaattcactggccg | SEQ ID NO: 22 |
| 2UProx1-F | ggccagtgaattagaactcggatcggccgcgtggaactac | SEQ ID NO: 23 |
| 2UProx1-R | aaggatagtaagctggcaaatgttgattgtctaactgcgttcttttgt | SEQ ID NO: 24 |
| 3GAL7-F | aagaacgcagttagacaatcaacatttgccagcttactatccttcttgaaaat | SEQ ID NO: 25 |
| | atg | |
| 3GAL7-R | AATTTTGGAGAAACGTCGGttttgagggaatattcaactgttttttttt | SEQ ID NO: 26 |
| | atcatgttga | |
| 4ApGGPPS-F | agttgaatattccctcaaaaCCGACGTTTCTCCAAAATTCATTTCA | SEQ ID NO: 27 |
| | ATTTTTTC | |
| 4ApGGPPS-R | tagaaacattttgaagctatTCAATTCTGCCTCCGACCAATGTAC | SEQ ID NO: 28 |
| 5TEF1-F | ATTGGTCGGAGGCAGAATTGAatagcttcaaaatgtttctactcctt | SEQ ID NO: 29 |
| | ttttactcttc | |
| 5TEF1-R | AGGAGGGAAAACAAAGGCATcttagattagattgctatgctttcttt | SEQ ID NO: 30 |
| | ctaatgagc | |
| 6ApCPS-F | gcatagcaatctaatctaagATGCCTTTGTTTTCCCTCCTCG | SEQ ID NO: 31 |
| 6ApCPS-R | gcataaatttttagttaaagggTCAGAAGTAACGGCGGGTATGGT | SEQ ID NO: 32 |
| | C | |
| 7DOWNrox1-F | CATACCCGCCGTTACTTCTGAccctttaactaaaaatttatgcatttg | SEQ ID NO: 33 |
| | gctcc | |
| 7DOWNrox1-R | tatgaccatgattacgccaagctgtttaacaaagcttaacattgtcaaattctt | SEQ ID NO: 34 |
| | cagg | |
| Cas9-F | TGTTAATACTTCTAACTATAgttttagagctagaaatagcaagttaaa | SEQ ID NO: 35 |
| | ataaggctag | |
| Cas9-R | TATAGTTAGAAGTATTAACAgatcatttatctttcactgcggagaagtt | SEQ ID NO: 36 |
| | tc | |

### Example 5 Construction of Eukaryotic Microorganism Expression System for CYP71A8t and CYP71D10t

Using Andrographis paniculata genome as a template, primer CPR-F/CPR-R was used to amplify ApCPR gene. Using S. cerevisiae genome as a template, primer P_{GAL7}-F/P_{GAL7}-R was used to amplify promoter P_{GAL7}, and primer PTEF1-F/PTEF1-R was used to amplify promoter P_{TEF1}. Using a nucleotide sequence of CYP71A8 as a template, primer CYP71A8t-F/CYP71A8t-R was used to amplify CYP71A8t. Using a nucleotide sequence of CYP71D10 as a template, primer CYP71D10t-F/CYP71D10t-R was used to amplify CYP71D10t. Using expression vector pY26-GDP-TEF as a template, primer pY26-F/pY26-R was used to amplify a linearized vector. The amplified products were recovered by ethanol precipitation. The promoter P_{GAL7}, promoter P_{TEF1}, CYP71A8t, CYP71D10t and linearized vector were subjected to Gibson assembly to obtain a recombinant vector. The recombinant vector was transferred into E. coli JM109 competent cells, spread on an LB solid medium containing 100 mg/L ampicillin, and cultured at 37° C. overnight. Positive clones were selected and plasmids were extracted. Correctly sequenced plasmids were transferred into the S. cerevisiae CW1006 constructed in Example 4, spread on YNB solid plates (containing 50 mg/L Leu, His and Trp), and cultured at 30° C. for 2-3 days. Positive clones were selected, and recombinant S. cerevisiae CW1006/pY26-P TEF1-CYP71A8t-P_{GAL7}-CYP71D10t was constructed (plasmid map shown in FIG. 5).

TABLE 4
Primers used for constructing pY26-PTEF1-CYP71A8t-PGAL7-CYP71D10t expression vector

| Primer | Sequence (5′-3′) | |
|---|---|---|
| CPR-F | GTAAAGGGGGSGGGGSGGGGSATGGATTCGCGGCTGGAG | SEQ ID NO: 37 |
| CPR-R | gaatgtaagcgtgacataacTCACCAAACATCCCTCAGGTATCG | SEQ ID NO: 38 |
| PGAL7-F | accaaacctctggcgaagaatttgccagcttactatccttcttgaaa | SEQ ID NO: 39 |
| PGAL7-R | GGGGGCAAGTTCTTGGTCATttttgagggaatattcaactgtttttttttat | SEQ ID NO: 40 |
| | catgttg | |
| CYP71A8t-F | agttgaatattccctcaaaaATGACCAAGAACTTGCCCCC | SEQ ID NO: 41 |
| CYP71A8t-R | cattttgaagctatgagctcAACGAACACACCCTCAGTATAGAACTTT | SEQ ID NO: 42 |
| | G | |
| PTEF1-F | CAAAGTTCTATACTGAGGGTGTGTTCGTTgagctcatagcttcaaaat | SEQ ID NO: 43 |
| | gtttctactcctt | |
| PTEF1-R | GAACTCCGGGGACGTTTCATactagttctagaaaacttagattagattgc | SEQ ID NO:44 |
| | tatgctttc | |
| CYP71D10t-F | ctaagttttctagaactagtATGAAACGTCCCCGGAGTTCC | SEQ ID NO:45 |
| CYP71D10t-R | ATCCATSCCCCSCCCCSCCCCCTTTACAGGCAAAGGGTTTTTCA | SEQ ID NO:46 |
| | ATTTGG | |
| pY26-F | ACCTGAGGGATGTTTGGTGAgttatgtcacgcttacattcacgcc | SEQ ID NO:47 |
| pY26-R | aaggatagtaagctggcaaattcttcgccagaggtttggtc | SEQ ID NO:48 |

### Example 6 Synthesis of 3,15,19-Trihydroxy-8(17),13-ent-labdadiene-16-oic Acid by Recombinant S. cerevisiae

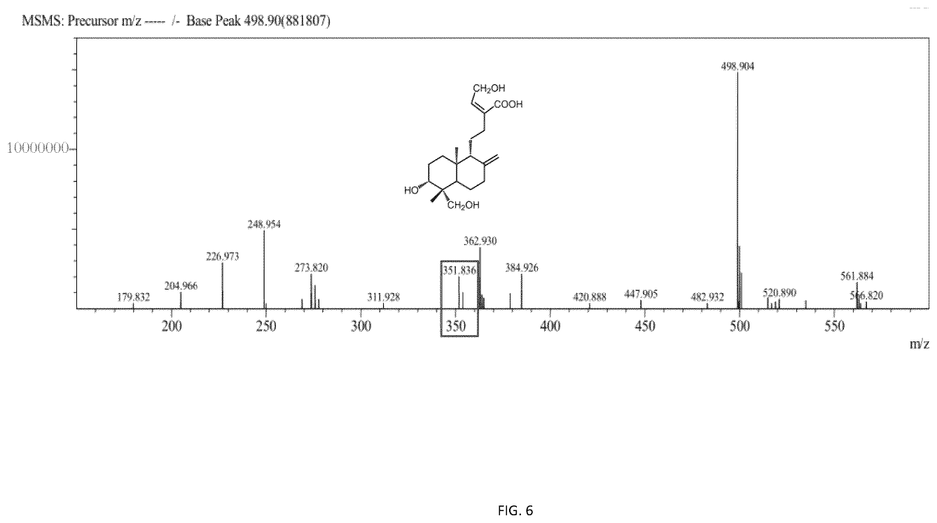

The recombinant S. cerevisiae CW1006/pY26-P_{TEF1}-CYP71A8t-P_{GAL7}-CYP71D10t constructed in Example 5 was streaked and inoculated into an LB medium containing 50 mg/L ampicillin, and cultured at 37° C. overnight. Single colonies were selected and transferred into an YNB medium (containing 50 mg/L Leu, His and Trp), and cultured at 30° C. and 220 rpm for 16-18 h. The culture was transferred to a fresh 25 mL YPD medium at a 1% (v/v) inoculation amount, and cultured at 30° C. and 220 rpm. After fermentation for 120 h, fermentation broth was collected, and a response value of 3,15,19-Trihydroxy-8(17),13-ent-labdadiene-16-oic acid was measured by LCMS-IT-TOF. The results were shown in FIG. 6. A response value of a product peak reached 1.9*106.

Although the present disclosure has been disclosed in exemplary embodiments above, it is not intended to limit the scope of the present disclosure. Those skilled in the art can make various changes and modifications within the spirit and scope of the present disclosure, and therefore the scope of protection of the present disclosure should be defined by the claims.

What is claimed is:

1. A recombinant Saccharomyces cerevisiae, comprising knockout of ROX1 and GAL80 genes on the genome; integrative expression of a GGPP synthase encoding gene and CPS diterpene synthase encoding gene; and free expression of CYP71A8 encoding gene CYP71A8t with N-terminal truncated by 32 amino acids, CYP71A10 encoding gene CYP71D10t with N-terminal truncated by 28 amino acids, and CPR encoding gene ApCPR;
wherein a nucleotide sequence of the CYP71A8 with N-terminal truncated by 32 amino acids is set forth in SEQ ID NO:3 and a nucleotide sequence of the CYP71D10 with N-terminal truncated by 28 amino acids being shown in SEQ ID NO:4; a nucleotide sequence of the ApCPR is set forth in SEQ ID NO:7; a nucleotide sequence of the GGPP synthase is set forth in SEQ ID NO:8; and a nucleotide sequence of the CPS diterpene synthase is set forth in SEQ ID NO:9.

2. The recombinant S. cerevisiae according to claim 1, wherein after the knockout of ROX1 site, the GGPP synthase encoding gene and the CPS diterpene synthase encoding gene are integrated at the ROX1 site.

3. The recombinant S. cerevisiae according to claim 1, wherein promoter P_{PGK1} is used for starting expression of the CPR encoding gene; promoter P_{TEF1} is used for starting expression of the CYP71A8t; and promoter P_{GAL7} is used for starting expression of the CYP71D10t.

4. The recombinant S. cerevisiae according to claim 1 3, wherein pY26 series vectors or pET series vectors are used as expression vectors.

5. A whole-cell catalyst, containing the recombinant S. cerevisiae according to claim 1.

6. A method for synthesizing 3,15,19-Trihydroxy-8(17),13-ent-labdadiene-16-oic acid, using the recombinant S. cerevisiae according to claim 1 or the whole-cell catalyst comprising the recombinant S. cerevisiae as a fermentation strain for fermentation to produce 3,15,19-Trihydroxy-8(17),13-ent-labdadiene-16-oic acid.
